(19) United States
(12) Patent Application Publication
(10) Pub. No.: US 2014/0221867 A1
(43) Pub. Date: Aug. 7, 2014

(54) WIRELESS PROBE FOR DENTAL ELECTROMYOGRAPHY

(71) Applicant: BTS S.p.A., Garbagnate Milanese (IT)

(72) Inventors: Fabrizio Renzulli, Milan (IT); Jacopo Costella, Milan (IT); Bruno Ros, Milan (IT); Enrico Fermi, Milan (IT)

(73) Assignee: BTS S.p.A., Garbagnate Milanese (IT)

(21) Appl. No.: 14/173,524

(22) Filed: Feb. 5, 2014

(30) Foreign Application Priority Data
Feb. 6, 2013 (IT) MI2013A000164

(51) Int. Cl. A61B 5/0488; A61B 5/00

(52) U.S. Cl. CPC A61B 5/0488; A61B 5/0015
USPC 600/546

(57) ABSTRACT

A wireless probe for electromyography to be used in dentistry, suitable for acquiring data even in the absence of a receiving unit, which is easily positionable onto and removable from the patient's face. The wireless probe preferably includes a first recording electrode and a first device provided with a second recording electrode.

### OBJECT OF THE INVENTION

The present invention concerns a wireless probe for electromyography to be used in dentistry, suitable for acquiring data even in the absence of a receiving unit, agilely positionable onto and removable from the patient's face

### STATUS OF THE ART

Electromyographs are traditionally equipped with a number of probes connected, via cables, to the recording devices used to record the electrical signals generated by the under test muscles. In order to overcome the drawbacks caused by the presence of said cables, the wireless technology has been applied to electromyographs too. Thanks to this new technology, the use of the electromyographs in dentistry became easier, in particular for the functional analysis of dental occlusions.

With reference to the electromyographs used in dentistry, electromyographs are known, equipped with 3 electrodes: 2 for recording purposes and 1 for the patient's bias. The recording electrodes are either fixed geometry electrodes, i.e. featuring a fixed interdistance between them or variable geometry ones, i.e. featuring a variable interdistance.

Such configuration of the electrodes entails a number of drawbacks including, amongst others, the difficulty found in applying to the patient at least three electrodes for every point to be examined, i.e. two electrodes for recording purposes and one electrode for patient's bias, which also makes the patient's movements less easy during the dental electromyographic test. In particular, remember that a patient undergoing a functional analysis to check for a possible dental occlusion via an electromyographic examination shall perform a mastication operation. It follows that making the patient's movements natural is one of the most important factors to get data that is as much representative as possible of the possible masticatory occlusal status of the patient.

In the electromyographs known so far, another drawback of the wireless electrodes consists in that they include a female knurled button to make it possible to apply the electrode to the patient by means of electrosensitive plasters equipped with male knurled buttons. A drawback of such a method used to apply the electrodes consists in that it exerts an annoying pressure onto the patient's face to make the knurled button portion of the electrode match the knurled button portion of the plaster.

### DISCLOSURE OF THE INVENTION

The present invention advantageously solves the drawbacks of the known devices via a wireless probe for dental electromyography equipped with a first recording electrode and a first device, preferably a container, equipped with a second recording electrode in which no patient's bias electrode is equipped. An advantage of the present invention is in that it is more compact with respect to the known devices; as a matter of fact, it makes it possible to apply two electrodes only to the patient for every probe and to use one probe only for every area to be examined. Remember that, for every dental electromyographic test, several areas of the patient's face and neck shall be examined. Therefore, the probe according to the present invention reduces the number of the electrodes used: remember that the known electromyographs use three electrodes for every area to be examined.

Another problem solved by the probe according to this invention concerns the need, in dental electromyographic studies, for repeating the electromyographic test several times during a long lapse of time, even several hours. Let's consider, for instance, a dental occlusion functional analysis and the study of the possible solutions to said occlusion. In these cases, the patient shall first undergo an electromyographic test without any restrictions and then he shall repeat the test, one or several different bytes. Such a procedure implies that the various specimens of the electromyographic probe shall be applied to the patient for several hours. Consequently, these probes shall have an autonomy of operation of several hours. If such probes don't have an autonomy of operation sufficient to cover the period of time necessary to perform the necessary cycle of tests, for instance to complete the above mentioned medical protocol, then the mentioned probes should be removed from the patient's face in order to be recharged and applied again onto the patient's face for the subsequent tests specified by the protocol.

A drawback of such a removal is obviously in that it extends the overall time necessary to perform the complete protocol, the patient being obliged to wait until every specimen of the probe is recharged. Another drawback consists in the difficulty for the operators to apply every specimen of the probe exactly in the same position where it had been applied in the previous test cycles.

The common experience tells that it is substantially impossible to place an object onto a surface whatsoever in the same position for two times.

In order to overcome this drawback, in the probe according to the present invention, at least the former device or the former electrode is provided with a battery. Said battery might also be equipped in both the first device and the first electrode.

Whether to equip either the former device or the former electrode with a battery rather than both, it depends on the autonomy specified for the probe to the detriment of the probe's weight, indeed. For complex cases requiring many tests and consequently long periods of time, a probe equipped with two batteries will certainly be preferable to the one-battery version, even though the probe's weight will be higher. For a patient, the inconvenience of having slightly heavier probes applied onto his face is compensated for by the possibility of preventing one or several specimens of the probe from having no sufficient energy for all the test duration, thus avoiding the risk of being obliged to remove one or all specimens of the probe in order for them to be recharged and then be put again in their places, a circumstance that would imply the re-positioning problems mentioned above. Different is the case of tests requiring shorter times for being performed; in this case, a one-battery probe should be adopted indeed, which would also make the presence of the probes on the face less tiresome, being their weight imperceptible. Therefore, the present invention provides at least one battery but a probe equipped with two or more batteries is also an object of the present invention to be covered by the patent.

Strictly related to the problems concerning the need for repeating the electromyographic test after several hours without removing the individual specimens of the probe from the patient's face as described above is the possibility of having the patient located far away from the receiving unit.

In the wireless electromyographs known so far, the patient shall be located at a given distance from the receiving unit because said receiving unit records the patient's muscle reactions under test, in real time. The perimeter within which the patient shall be located depends on the electromyograph's wireless antenna range. However, this restriction is a drawback in that the patient is obliged to stay close to said receiving unit, thus obliging all professionals involved in the test and in the study of the possible solutions to the patient's pathology to crowd in the perimeter determined by the range of the mentioned wireless antenna.

In order to overcome this drawback, in the probe according to this invention the first device is equipped with first storage means to store the signals from the first electrode and from the second electrode. The data stored by said means might be downloaded onto the receiving unit at a time subsequent to that when the test is carried out, thus enabling the medical doctor to analyze it in due time, which is an evident advantage for both the medical doctor and the patient.

Note that another advantage resulting from the presence of said first storage means consists in the possibility of starting an electromyographic test even in the case that the receiving unit is temporarily out of order or in the case of a temporary failure of the electrical power system that supplies power to the receiving unit.

As described above, the probe according to the present invention comprises, amongst others, a first electrode and a first device. These two elements shall be electrically connected to each other to enable the probe to operate. Said elements shall also be applied onto the patient's face, whose form is not flat. To meet these two requirements, the two elements are advantageously electrically connected to each other by an electrical cable featuring elastic properties, thus making the application of the first electrode and of the first device to the face, easier.

As said above, the probe according to the present invention shall be applied onto the patient's face. In order to prevent pressures from being exerted onto the patient's face while the probe is applied, as with the known probes, the probe according to the present invention is equipped with magnets suitable for being coupled with appropriate electrosensitive plasters. Said magnets are equipped in the first electrode and in the first device to make it possible the coupling of said plasters having a first face coated by an adhesive material to adhere to the patient's face and a second face provided with a metal surface to make it possible the coupling with the mentioned electrode and with the mentioned device.

As described above, the operating autonomy of the probe (i.e. the duration of its electrical charge) is a very important factor, along with its miniaturization, which should be as high as possible. In order to maximize such a miniaturization, the first device also comprises recharging means suitable for recharging one or several batteries of the probe upon placing the latter in the receptacle reserved for this purpose in the recharging device. In order to maximize the duration of the mentioned electrical charge, said probe is provided with an on/off switch complete with a magnetic switch-on/switch-off device. Said on/off switch deactivates the probe whenever this one is put in the receptacle of said probe recharging device. This way, the risk that an inattentive operator might leave the probe switched on even when not necessary, is avoided. Likewise, said on/off switch activates the probe upon taking it out of the receptacle of the recharging device complete with magnetic device, thus making said probe ready for use even in the case the operator forgets to activate it before applying it onto the patient. Finally, said on/off switch might also operate in activating or deactivating the probe in cases other than those mentioned above, through a direct manual operation by the operator onto said on/off switch.

### DESCRIPTION OF THE FIGURES

FIG. 1 shows a perspective view of the wireless probe assembly according to the present invention.

FIG. 2 shows a plan view of a plaster suitable for being applied onto the cutis of the patient and coupled with the mentioned probe.

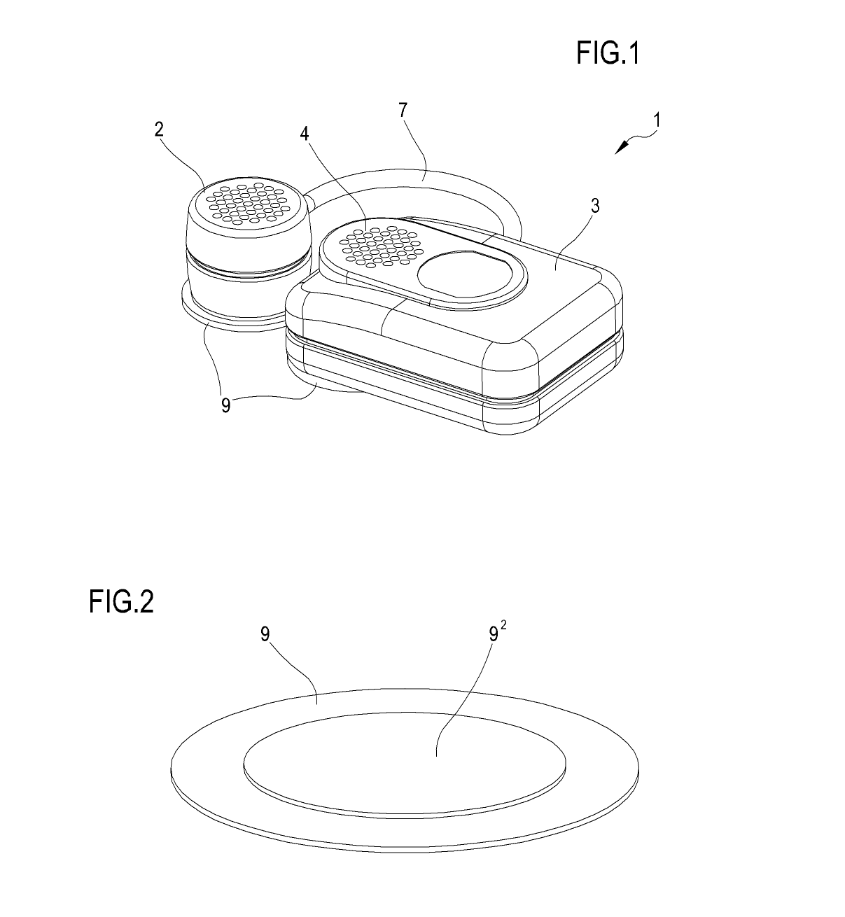

FIG. 1 shows a perspective view of the wireless probe (1) according to the present invention for dental electromyography. This figure shows a first recording electrode (2) and a first device (3) provided with a second recording electrode (4). The first recording electrode (2) and/or the first device (3) are equipped with a battery, not shown in the drawing. Said first recording electrode (2) and the first device (3) are also equipped with magnets (8), not shown in the figures, for their coupling with the plaster (9), better illustrated in FIG. 2. The first recording device (3) is provided with means (6), not shown in the figures, suitable for storing the signals coming from the first electrode (2) and from the second electrode (4), and recharging means (12), not shown in the figures, suitable for recharging the battery (5) of the probe (1). The first electrode (2) is connected to the first device (3) via an electrical cable (7) featuring elastic properties. The probe (1) according to the present invention is also provided with at least one magnetic on/off switch (10), not shown in the figures, suitable for deactivating the probe (1) upon accommodating it in the receptacle of the recharging device of said probe (1), not shown in the figures, provided with a magnetic device (11), not shown in the figures. Said on/off switch (10) is also suitable for activating the probe upon removing it from the receptacle of the recharging device of said probe (1), but it is also suitable for activating or deactivating the probe (1) upon a direct manual operation by the operator onto said on/off switch (10).

FIG. 2 shows a plan view of an electrosensitive plaster suitable for being applied onto the cutis of the patient and coupled with said probe, in which the plaster (9) comprises a first face (9¹), not shown in the figure, coated with an adhesive material, an a second face (9²) coated with a metal surface to make it possible the coupling of said plaster (9) with the first recording electrode (2) and with the first device (3).

1. A wireless probe for dental electromyography,
characterized in that it comprises a first recording electrode and a first device provided with a second recording electrode.

2. A wireless probe for dental electromyography according to claim 1, characterized in that the first device is provided with at least one battery.

3. A wireless probe for dental electromyography according to claim 1, characterized in that the first device is provided with means to store the signals coming from the first electrode and from the second electrode.

4. A wireless probe for dental electromyography according to claim 1, characterized in that the first device is provided with charging means suitable for recharging the battery of the probe.

5. A wireless probe for dental electromyography according to claim 1, characterized in that the first electrode is connected to the first device by an electrical cable featuring elastic properties.

6. A wireless probe for dental electromyography according to claim 1, characterized in that the first electrode and the first device are provided with magnets suitable for coupling with an electrosensitive plaster suitable for being applied onto the patient's cutis.

7. A wireless probe for dental electromyography according to claim 6, characterized in that said electrosensitive plaster comprises a first face coated with an adhesive material and a second face coated with a metal surface.

8. A wireless probe for dental electromyography according to claim 3, characterized in that the probe is provided with at least one magnetic on/off switch.

9. A wireless probe for dental electromyography according to claim 8, characterized in that said on/off device is provided with a magnetic device suitable for deactivating the probe upon placing it in the receptacle of the recharging device of said probe.

10. A wireless probe for dental electromyography according to claim 8, characterized in that said on/off switch is provided with a magnetic device suitable for activating the probe upon removing it from the receptacle of the recharging device of said probe.
